(19) United States
(12) Patent Application Publication
(10) Pub. No.: US 2015/0112209 A1
(43) Pub. Date: Apr. 23, 2015

(54) CARDIO-POSTURAL ASSESSMENT SYSTEM

(71) Applicants: Andrew P. BLABER, Port Moody (CA); Kouhyar TAVAKOLIAN, Burnaby (CA); Alexandre LAURIN, St-Lambert (CA)

(72) Inventors: Andrew P. BLABER, Port Moody (CA); Kouhyar TAVAKOLIAN, Burnaby (CA); Alexandre LAURIN, St-Lambert (CA)

(21) Appl. No.: 14/522,457

(22) Filed: Oct. 23, 2014

Related U.S. Application Data
(60) Provisional application No. 61/894,866, filed on Oct. 23, 2013.

(51) Int. Cl. A61B 5/0205; A61B 5/0488; A61B 5/11; A61B 5/0245

(52) U.S. Cl. CPC A61B 5/0205; A61B 5/0245; A61B 5/0488; A61B 5/1102; A61B 5/1116
USPC 600/483

(57) ABSTRACT

An exemplary cardio-postural assessment system (CAS) may desirably provide for continuous cardiovascular and postural data monitoring and assessment of a subject during standing. One such CAS may provide continuous cardiovascular and postural data monitoring using a non-invasive weight-scale platform. Another such CAS device may allow for an assessment of balance/posture control, posture muscle activation, and cardiovascular function components simultaneously and provide detailed output as to the proportion each area contributes to the cardio-postural stability of an individual.

### CROSS-REFERENCE TO RELATED APPLICATIONS

This application is related and claims priority to U.S. Provisional Patent Application Ser. No. 61/894,866 filed Oct. 23, 2013, which is hereby incorporated herein by reference in its entirety.

### TECHNICAL FIELD

The present invention relates generally to systems for cardiovascular and postural assessment. More specifically, the present invention relates to cardio-postural assessment systems which are adapted to monitor both cardiovascular and postural control of a subject.

### BACKGROUND OF THE INVENTION

Currently the monitoring of balance/posture control, posture muscle activation, and cardiovascular function of a subject or patient are performed with several different devices. With patients experiencing falls the diagnosis of possible related balance, posture, and cardiovascular disorders relies on a series of tests conducted outside the Doctor's office and requires the efforts of several specialists and typically multiple different devices and/or equipment to determine if the subject or patient's problems are neuromuscular, vestibular, or cardiovascular.

Accordingly, there remains a desire for improved cardio-postural assessment systems and methods for their use in assessment, diagnosis, monitoring and treatment that address some of the limitations of the existing systems known in the art.

### SUMMARY OF THE INVENTION

According to one aspect, it is an object of the present invention to provide a cardio-postural assessment system that addresses some of the limitations of the prior art.

According to another aspect, a single-unit physiology monitoring device designed to simultaneously assess cardiovascular and postural control may be provided.

According to a further aspect, one embodiment of the present invention may desirably provide a reliable portable device for assessment of cardio-postural health that would provide all the required inputs to an exemplary cardio-postural model and output indices such as for personal, professional and clinical use. In one such embodiment, these indices may be based on a robust analytical model of the cardiovascular and postural control systems through integrated system modeling and mathematical analysis approaches (such as further described herein).

According to one embodiment of the present invention, a cardio-postural assessment system is provided, comprising:

a non-invasive force platform comprising at least one force sensor;

at least one pair of electromyography sensors;

at least one pair of electrocardiography sensors;

at least one ballistocardiography sensor; and

an electronic processor connected to said at least one force sensor, said electromyography sensors, said electrocardiography sensors, and said ballistocardiography sensor and adapted to receive signals therefrom, to calculate a center of pressure from signals received from said force sensor, an electromyogram from signals received from said electrocardiography sensors, an electrocardiogram from signals received from said electrocardiography sensors, and a ballistocardiogram from signals received from said ballistocardiography sensor, and further to analyze at least a plurality of said center of pressure, electromyogram, electrocardiogram and ballistocardiogram to generate at least one output signal corresponding to a cardio-postural physiological model.

In a further embodiment of the present invention, a method for non-invasive cardio-postural assessment may be provided, the method comprising:

receiving signals from force, electromyography, electrocardiography and ballistocardiography sensors in communication with a subject under assessment;

calculating a center of pressure from signals received from said force sensor, an electromyogram from signals received from said electrocardiography sensors, an electrocardiogram from signals received from said electrocardiography sensors, and a ballistocardiogram from signals received from said ballistocardiography sensor;

analyzing at least a plurality of said center of pressure, electromyogram, electrocardiogram and ballistocardiogram to define discrete interaction events between signals and interaction strength of said interaction events;

analyzing said interaction events to determine time overlapping pairs of interaction events;

analyzing a plurality of said time overlapping pairs of interaction events to determine a degree of phase lock correlation between said pairs of interaction events within at least one frequency band;

determining a residual time delay from a phase difference of said pairs of interaction events for a plurality of single wavelengths;

determining an overall time delay for said pairs of interaction events from a consecutive sequence of said residual time delays over a plurality of said wavelengths;

determining a causality between said interaction events for each said pair of interaction events from said overall time delay for said pair; and

determining a strength of interaction between each said pair of interaction events from a maximum mean gain of said time delay for said pair of interaction events.

In another embodiment of the present invention, a method for non-invasive cardio-postural assessment as provided above may additionally comprise outputting at least one of said time delay, causality and strength of interaction between at least one pair of cardio-postural parameters of a cardio-postural model to an operator.

Further advantages of the invention will become apparent when considering the drawings in conjunction with the detailed description.

### BRIEF DESCRIPTION OF THE DRAWINGS

The system according to several embodiments of the present invention will now be described with reference to the accompanying drawing figures, in which:

FIG. 1 is a schematic view of a Cardio-postural Assessment System (CAS) comprising a non-invasive weight-scale platform, according to an embodiment of the present invention.

FIG. 2 is a schematic view of a physiological model of cardio-postural interactions with both feedforward and feedback pathways, according to an embodiment of the invention.

FIG. 3 is a schematic view of a COP (center of pressure) circuit comprising an exemplary analog circuit (but digital and other electronic versions are possible in other embodiments and are expressly contemplated in the present specification), according to a further embodiment of the present invention.

FIG. 4 is a schematic view of an ECG (electrocardiogram) circuit comprising an exemplary analog circuit (but digital and other electronic versions are possible in other embodiments and are expressly contemplated in the present specification), according to an embodiment of the invention.

FIG. 5 is a schematic view of an EMG (electromyogram) circuit comprising an exemplary analog circuit (but digital and other electronic versions are possible in other embodiments and are expressly contemplated in the present specification), according to an embodiment of the invention.

FIG. 6 is a schematic view of a BCG (ballistocardiogram) circuit comprising an exemplary analog circuit (but digital and other electronic versions are possible in other embodiments and are expressly contemplated in the present specification), according to an embodiment of the invention.

FIG. 7 is a graphical view of exemplary measured EMG and BP signals as well as an exemplary extracted beat-by-beat systolic blood pressure (SBP) series from a representative exemplary subject (panel A), according to an embodiment of the present invention, and a corresponding exemplary time-frequency map of wavelet coherence between EMG and SBP (panel B). An exemplary cone of influence (COI) is designated by the shaded area and the thick black contour indicates an exemplary statistical threshold of significant coherence. Exemplary electromyography (EMG); blood pressure (BP); systolic blood pressure (SBP); low frequency (LF); very low frequency (VLF); cone of influence (COI); total area outside of the COI in LF (VLF) band (A_{LF} (A_{VLF})); and total area with significant coherence outside of the COI in LF (VLF) band (A_{SC-LF} (A_{SC-VLF})) are also shown, according to one embodiment of the invention.

FIG. 8 shows Venn diagrams of percentage of area with phase lock (% PL) in both LF and VLF bands using cardiac output (CO) as the cardiovascular variable, according to an embodiment of the present invention, where EMG=Electromyography; CO=cardiac output; and COPr=overall center of pressure.

FIG. 9 shows a graphical view of the exemplary behaviour of residual time delay with respect to wavelength with a time delay of 5, according to an embodiment of the present invention. In one such embodiment, the change to null slope as wavelength becomes greater than 10 may be observed. In another embodiment, it may be impossible to visually identify linear behaviour for wavelengths smaller than some exemplary threshold, a, especially in a noisy signal. In a further embodiment, a similar exemplary graph, but with all signs of residual time delays being changed may result from Δt<0.

FIG. 10 is a graphical view illustrating an exemplary systematic error in measuring time delay using phase difference, according to an embodiment of the present invention.

FIG. 11 is a graphical view illustrating an exemplary residual time delay versus wavelength in a peak-to-nadir scenario, according to an embodiment of the present invention.

FIG. 12 is a graphical view illustrating an exemplary individual Δt vs. λ A graph, according to an embodiment of the invention. In such view, blue points are in an exemplary LF region while green points are in an exemplary HF region, and the vertical dotted line indicates an exemplary cut-off. The blue and green horizontal dotted lines represent best least-mean-absolute-error regressions and regions outside the domain of definition are shaded, in one embodiment.

FIG. 13 is a graphical view illustrating exemplary autonomic nervous system time delay according to an embodiment of the invention, where exemplary bins in the parasympathetic nervous system region have width 0.3 s and start at 0.15 s. Exemplary bins in the sympathetic nervous system region have width 1 and are centered at integers. In FIG. 13a) Population data (N=18) is shown: time delays are from individual means where an instance represents one participant. In FIG. 13b) Data from one participant is shown: an instance represents a 10 s analysis period in which a time delay was measured. Calculated mean PNS delay for the population was 0.90±0.31 s, mean gain of 32.6 ms/mmHg Mean SNS delay for the exemplary population was 6.8±1.5 s, mean gain of 13.7 ms/mmHg

### DETAILED DESCRIPTION

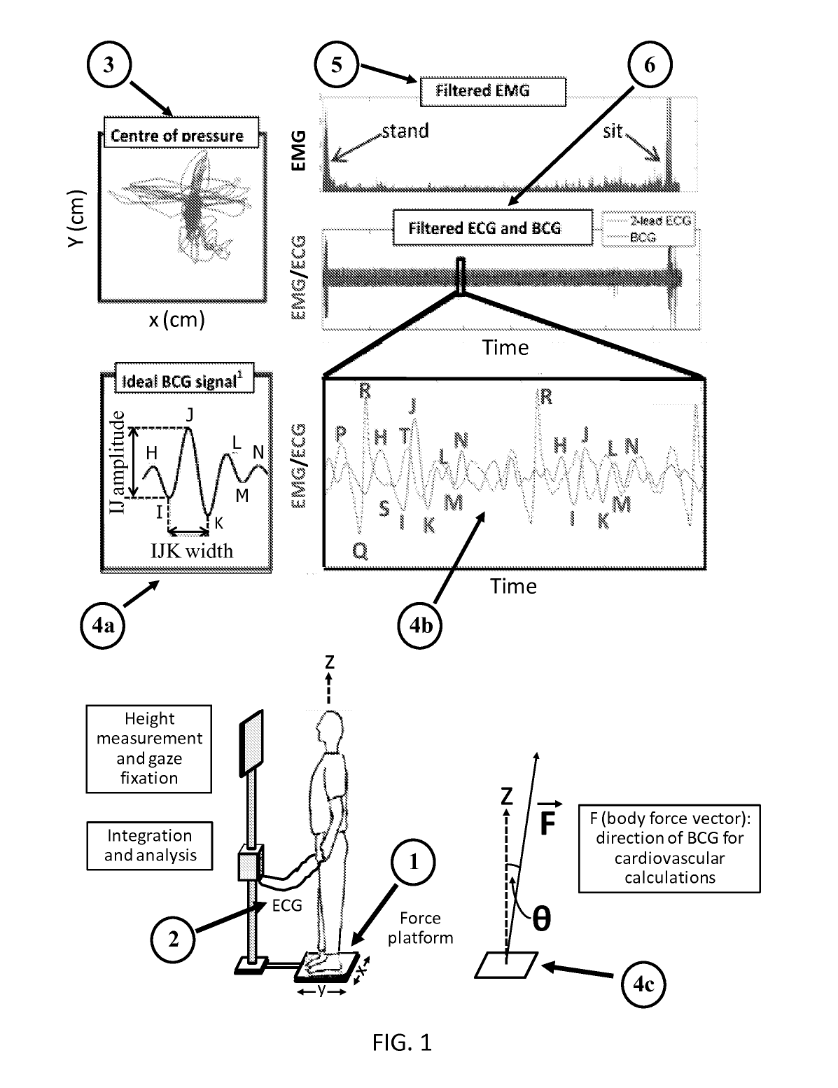

In one embodiment of the present invention, an exemplary cardio-postural assessment system (CAS) may desirably provide for continuous cardiovascular and postural data monitoring and assessment of a subject during standing. In one such embodiment, the CAS may provide continuous cardiovascular and postural data monitoring using a non-invasive weight-scale platform according to an embodiment of the present invention, such as is shown in FIG. 1, for example. In another embodiment of the invention, a device such as is described herein, may allow for an assessment of balance/posture control, posture muscle activation, and cardiovascular function components simultaneously and provide detailed output as to the proportion each area contributes to the cardio-postural stability of an individual.

According to an embodiment of the invention, the use of an exemplary cardio-postural assessment device and a suitable methodology, such as described herein, may desirably allow for an earlier prescription of treatment and streamline the referral of patients to the appropriate specialists. Furthermore, according to another embodiment, such a device may desirably enhance the tracking of progress of the patient during treatment/recovery. According to a further embodiment, a desirably small robust device may be provided, such that it could be used easily at home and with an internet connection data could be relayed to a Doctor's office, Health care, physiotherapy or Sports clinic, for example.

According to one embodiment, a target application of an exemplary cardio-postural assessment device is for one or more of: Home use (self monitoring), Physiotherapy and fitness centers, as well as Clinical assessment in Family doctor offices and Sports Medicine clinics. A modified Home device can also be used as an eHealth device with transmission capabilities to a Doctor, hospital, or other healthcare provider's office, for example.

### I. Hardware

In one embodiment of the invention, a simple short (10-15 s) measurement of BCG with an integrated electrocardiogram enables the extraction of information regarding the cardiovascular dynamics and estimate hemodynamic parameters for a subject or patient, such as stroke volume and cardiac output. Simultaneous measurement of changes in center of pressure of the feet along with electromyography of the leg muscles from the weight scale (force plate) over 1 to 5 minutes will provide information on the components of the cardio-postural system that can be used to diagnose a large number of health problems, including but not limited to: hemodialysis, falls, and after effects and recovery from concussion. In another embodiment, further associations with neurological and cardiovascular disorders such as with but not limited to Diabetes, pure autonomic failure, Parkinson's can be characterized.

In further detail, referring to one embodiment of the invention, such as is shown in FIG. 1: Schematic of cardio-postural assessment system (CAS): The force plate (1) records the center of pressure (3) from force sensors attached to the scale (x and y direction) and also the Ballistocardiogram (4a) from the same sensors (z direction) which is corrected for the body direction using COP vector analysis (4c) and Electromyogram (5). The EMG data is collected from electrodes embedded in the surface of the scale; there is one electrode for each foot. Each electrode is divided up in to multiple segments in the form of an array. In one embodiment, the device will search for the combination of segments that provide the best EMG signal from the person standing on the device. In an exemplary embodiment, the ECG electrodes held in the hand (2), record lead I of Electrocardiogram [note: the leads may also be from one hand and opposite foot, both feet, or attached to any position across the heart axis to obtain a signal] (4). Description of data according to an exemplary embodiment: (3)—Centre of pressure (COP) is calculated from force data; the X axis is Medial-lateral (ML: left-right), the y axis is anterior-posterior (AP: front to back). In the last 100 s the subject voluntarily swayed ML then AP. (5)—Filtered electromyography (EMG) data. Note large activation during transition from standing and sitting as well as increased activity during active sway. (6) Electrocardiogram (ECG, blue) and ballistocardiogram (BCG, red). (4a) Example of a BCG signal, redrawn from Starr et al. (1939) The named features of the BCG used to analyze the signal in the literature are shown. (4b) Expanded view of ECG and BCG signal with ECG and BCG signal features are labeled.

In one embodiment of the present invention, the cardio-postural assessment system may comprise an electronic processor adapted to receive signals from the force plate 1 (and its force sensors), ballistocardiography sensor(s) (which may comprise the same force sensors in the force plate, or one or more force sensor and/or accelerometer oriented in a vertical or z direction with respect to the force plate 1, such as is shown in COP vector analysis 4c), electromyography sensors (such as incorporated in the surface of force plate 1 in one embodiment, or by separate electromyography electrodes in another embodiment), and electrocardiography sensors such as electrocardiography electrodes or leads 2. In one such embodiment, the electronic processor may comprise one or more electronic circuits such as but not limited to:

an exemplary COP (center of pressure) circuit such as to calculate a center of pressure of a user or patient standing on the force plate 1 and/or to record a graphical or vector recording of changes in a center of pressure of a user or patient over an assessment time interval;

an exemplary ECG circuit such as to calculate an electrocardiogram and/or record a graphical or other suitable recorded ECG signal over an assessment time interval;

an exemplary EMG circuit such as to calculate an electromyogram and/or record a graphical or other suitable recorded EMG signal over an assessment time interval; and

an exemplary BCG circuit such as to calculate an ballistocardiogram and/or record a graphical or other suitable recorded BCG signal over an assessment time interval, for example.

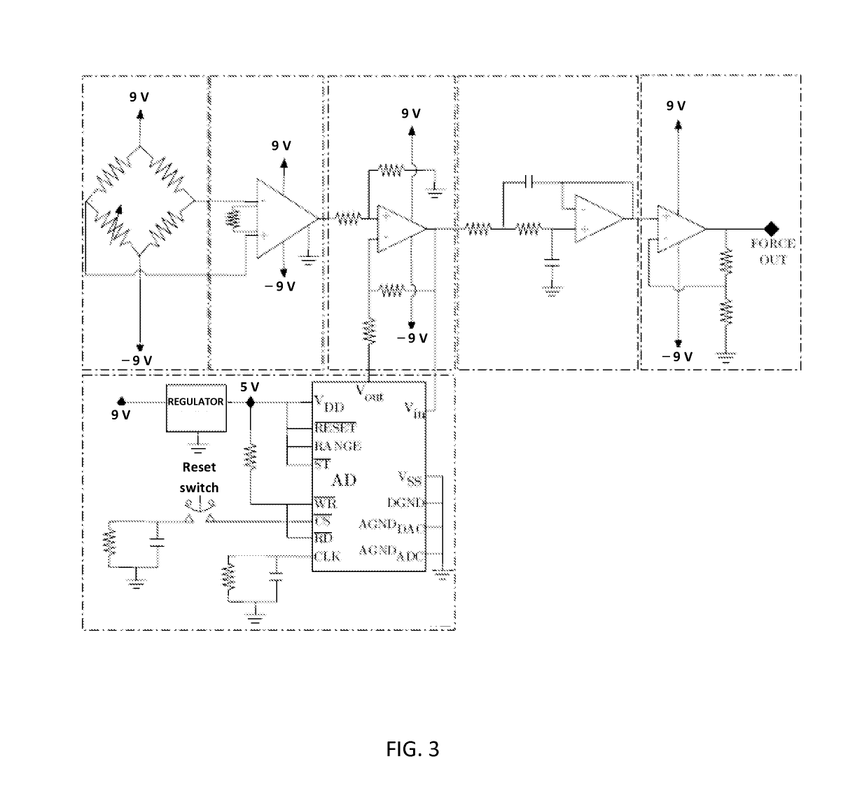

In a particular such embodiment, an exemplary COP circuit may comprise an exemplary analog circuit such as illustrated in FIG. 3, for example. In another embodiment, a suitable digital or other (such as mixed analog and digital circuits for example) electronic COP circuit may be utilized and is expressly contemplated in the present specification.

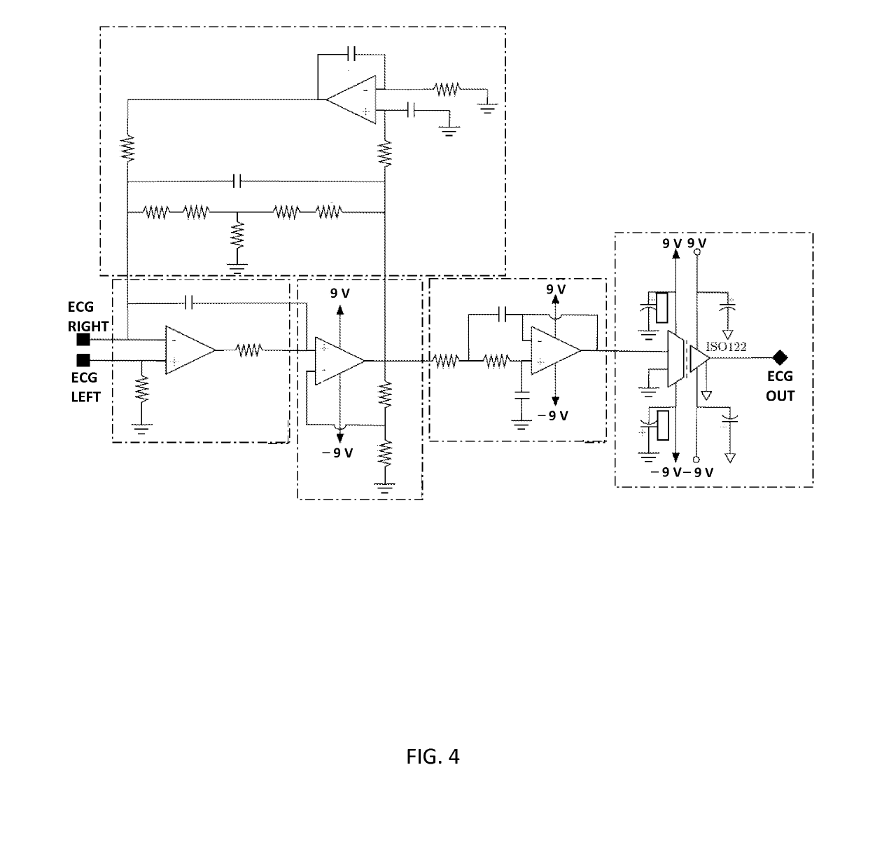

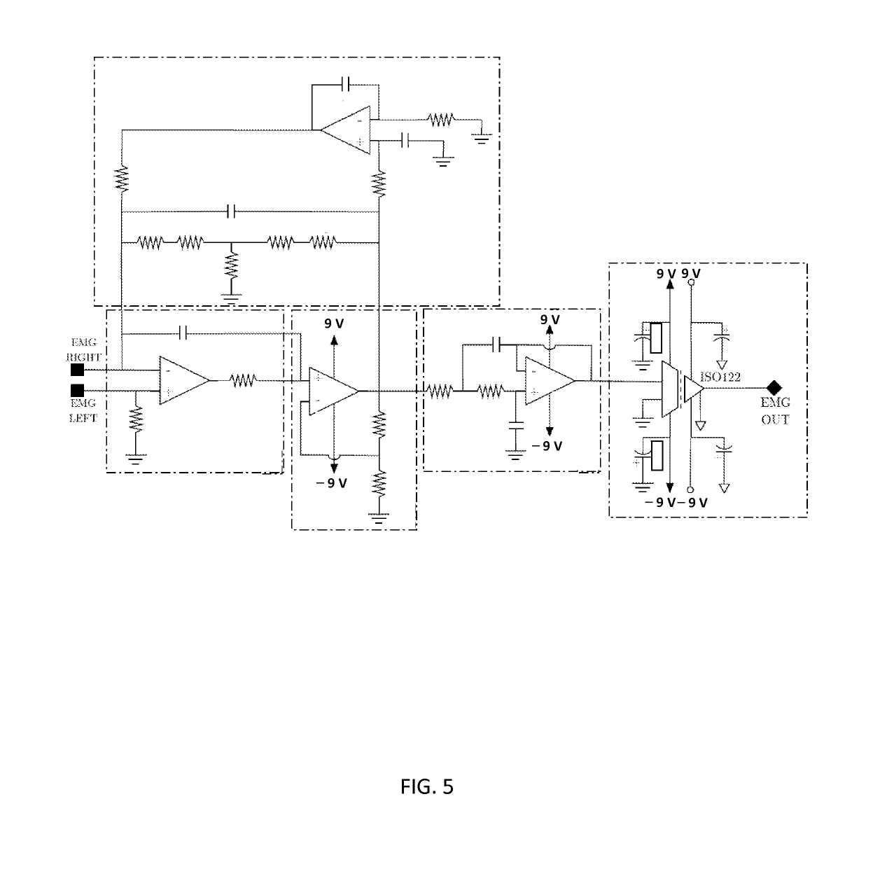

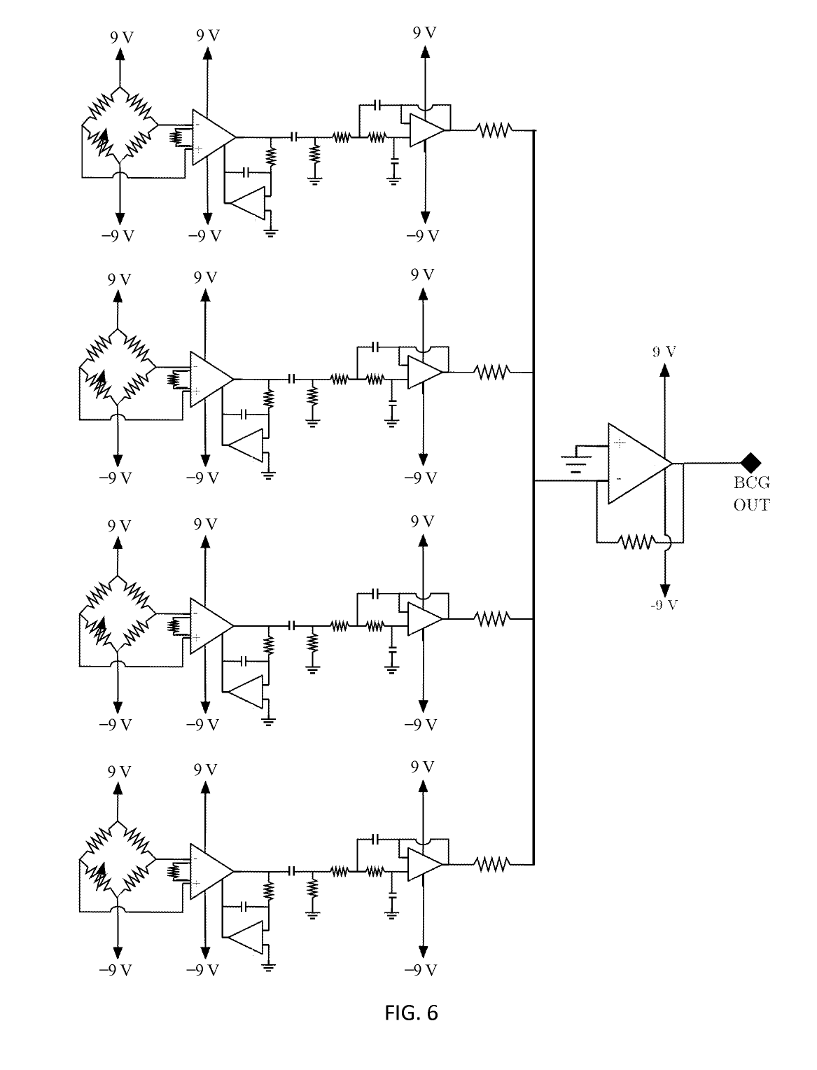

Similarly, in one embodiment exemplary ECG, EMG and BCG circuits may comprise one or more exemplary analog ECG, EMG and BCG circuits such as illustrated in FIGS. 4, 5 and 6, respectively, for example. In another embodiment, one or more suitable digital or other (such as mixed analog and digital circuits, for example) electronic circuit may be utilized and are expressly contemplated in the present specification.

In yet a further optional embodiment, a cardio-postural assessment system may comprise an electronic processor comprising combined and/or general purpose processing circuits to provide the functionality of one of more of a COP, ECG, EMG and BCG circuits, for example.

### II. Signal Processing and Cardio-Postural Assessment

In one embodiment of the present invention, once the signals from a suitable cardio-postural assessment device have been detected they are stored in a digital format, such as at sample rate greater than or equal to 250 Hz. Signal specific filters may be used to further process the data so that it can be input into the cardio-postural analysis. In one embodiment, this analysis is EVENT BASED rather than continuous.

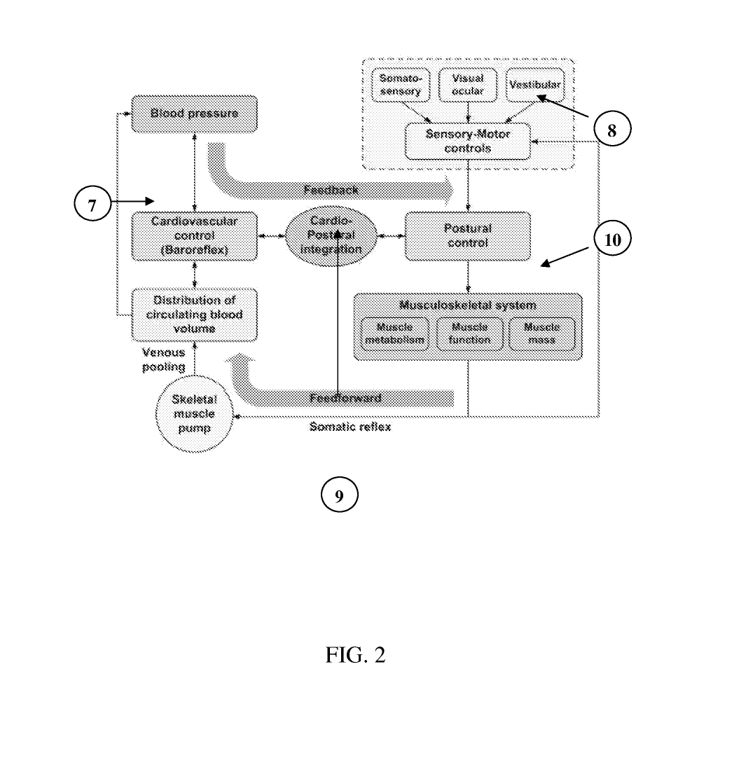

In further detail, referring to one embodiment of the invention, such as is shown in FIG. 2: Illustration of the Cardio-postural components associated with mechanisms that prevent syncope: (7)—cardiovascular component of the regulation of perfusion pressure in the brain (blood volume, heart rate and vascular resistance, cerebral autoregulation; (8)—sensory motor input components related to postural control; (9)—cardio-postural integration: hypothesized to occur at the level of the baroreflex to activate skeletal muscle pump through postural control; (10)—factors associated with muscle component of skeletal muscle pump strength.

According to one aspect of the present invention, a process is described to provide the strength (gain) and degree of interaction (overlap) of the components of the cardio-postural system for monitoring and diagnostic purposes. Furthermore, in one embodiment, a method to determine the individual time delay(s) between the input (eg: Cardiac Output from BCG) and EMG activation is provided, which are also of importance in assessing neurological and physical disorders. [NOTE: this process is not limited in its scope of application to just the cardio-postural system, but can be applied to any multiple input-output control system.]

According to one embodiment of the present invention, a method of analysis is provided comprising a specific EVENT BASED analysis process as provided below:
1. It describes the determination of interaction EVENTS through “PHASE LOCK.”
2. It describes how these identified EVENTS can be assigned a specific interaction strength.
3. It describes how timings of paired events can be linked through vertical time analysis of the interactions.
4. It describes how this Venn diagram can be used to determine how much interaction occurs and what components are solely due to one pair of interaction (eg. just muscle pump in a cardiovascular reflex).
5. It translates phase lock into time delay for a single wavelength.
6. It explains how to graphically present this time delay as a “funnel graph” to facilitate interpretation.
7. It explains how to interpret the obtained graph.
8. It explains how to obtain overall time delay(s) from the interpretation.
9. It is proposing a way to determine causality
10. Shows an example of time-delay for baroreflex.
   1. Determination of Interaction EVENTS through “PHASE LOCK:

In one embodiment, use of discrete time, phase and gain analysis of the data is performed to get cardio-postural related interactions between pairs of signals and determine the overlap in control mechanisms. Examples of time based analyses with phase and gain information are wavelets and Hilbert transforms, but other existing or not yet existing methods can be used.

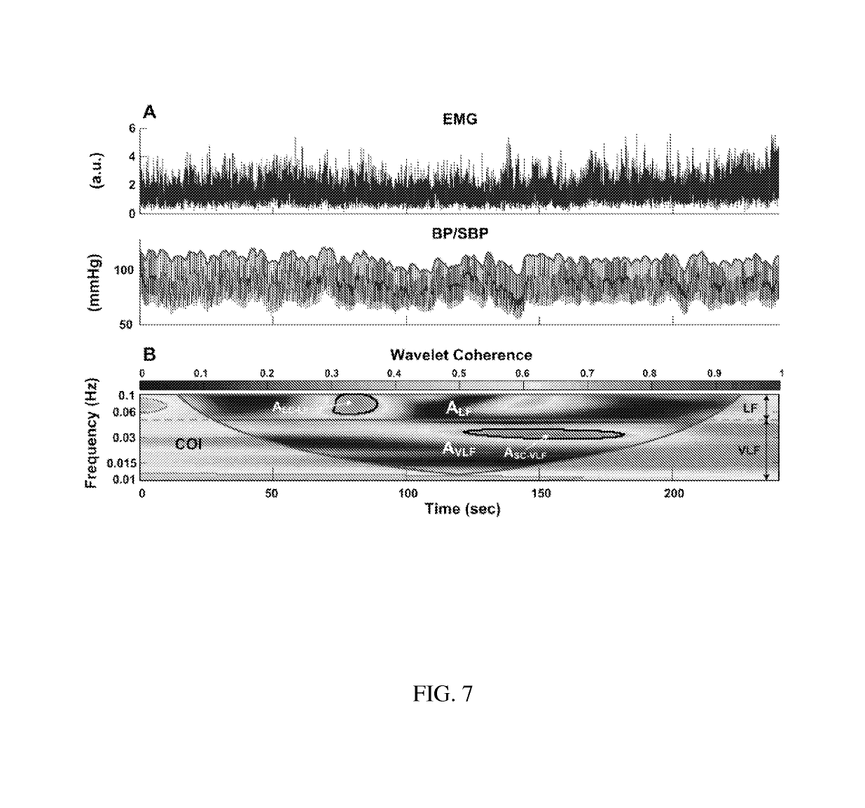

In a particular embodiment, an example of wavelet based coherence analysis between two signals (systolic blood pressure and calf muscle EMG) is shown in FIG. 7. In this exemplary case we have chosen two frequency regions (LF, VLF) in areas proposed to be related to skeletal muscle pump. Specifically chosen wavelets are used to find the phase, gain and coherence of the interaction between the two signals. In one embodiment, however, this analysis on gives general information of the interaction between the two signals at any given time, NOT specific event timing such as discrete muscle pump activation.

In a particular embodiment, once the time related phase and gain information have been obtained from the individual signals, pair wise comparisons are made. EVENTS are described as regions where, with adequate wavelet (or other) analysis of 2 signals, however you want to define it, can provide phase information. PHASE LOCK is defined the region, at any frequency, where the phase difference between the two signals does not significantly change (i.e the derivative of the phase difference is not different from zero). An EVENT is defined as any time segment related to physiological rationale where PHASE LOCK occurs. In one embodiment, for the cardio-postural analysis we have defined this as a time corresponding to 3 or more heart beat intervals (determined from mean heart beat interval from signals series).

### 2. EVENTS are Assigned a Specific Interaction Strength.

In one embodiment, the cross-wavelet (or other paired analysis) gain is used to represent the interaction strength.

3. Paired EVENTS are Linked through Vertical Time Analysis of the Interactions.

In one embodiment, once EVENTS have been located, the time series data are scanned for regions in which multiple paired EVENTS are observed. From this, a time related table of overlap of paired EVENTS is populated for graphical presentation and percentage estimation of overlap. This can be then related to the cardio-postural model (or any other type of multiple systems model).

### 4. Venn Diagram Presentation of Multiple Pairs of Interactions.

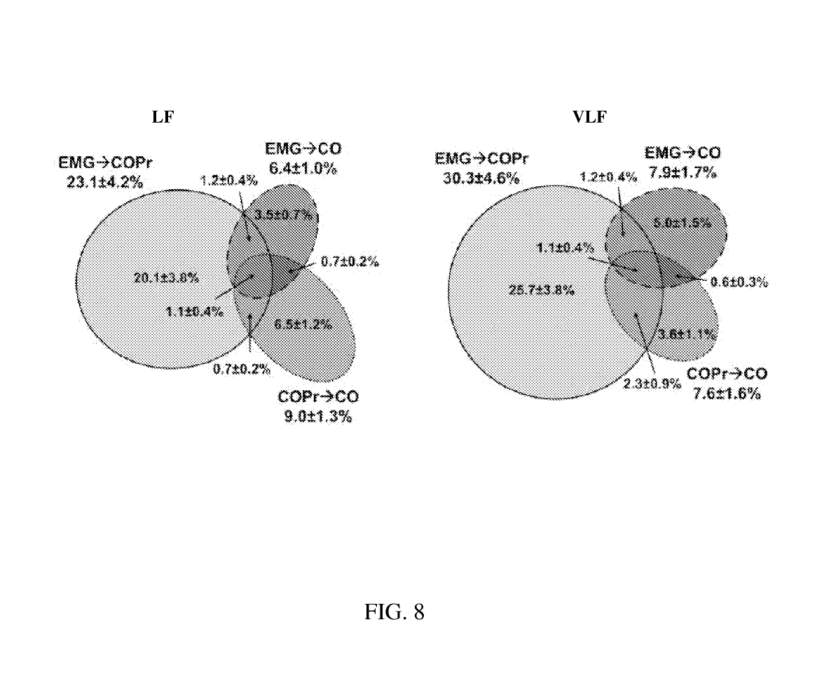

In one aspect, data from the time-interactions table are then constructed into a Venn diagrams of percent PHASE LOCK (% PL) in both LF and VLF bands to provide representation of the relationship between the individual paired components (FIG. 8). Each Venn diagram is composed of three ellipses, each of which represents the amount of interactions between a pair of signals (i.e., EMG and COPr, EMG and CO, and COPr and CO).

5. Translation of Phase Lock into Time Delay for a Single Wavelength.

In one embodiment, any suitable known methods that pick up phase lock will also give you phase difference Δφ in radians. In a particular embodiment, this may be a problem if a causal relationship that exists between two periodic phenomena depends on a constant time delay, Δt, instead of a phase delay. If we have phase difference for wavelengths that are larger than 2Δt, then their residual time delay will be exactly Δt. In other words, for λᵢ>2Δt,

Δt=Δti:

Empirically, one would obtain Δti's from Δφ's using:

$$\frac{\cdot Δ {φ}_{i} \cdot {λ}_{i}}{2 π} = Δ {t}_{i} ,$$

### 6. Graphical Presentation of Time Delay as a “Funnel Graph” to Facilitate Interpretation.

According to one embodiment, a plot of Δti versus λi. is used. From the origin (0, 0) the outer data boundaries of the graph expand along the line of Δ=±2Δt.

### 7. Interpretation of the Funnel Graph.

In one embodiment, two types of delay interactions may exist; one in which there is a positive interaction (i.e. when one increases the other increases: peak-to-peak) and in which there is a negative interaction (i.e. when one increases the other decreases: peak-to-nadir).

### i) Peak-to-Peak Time Delay:

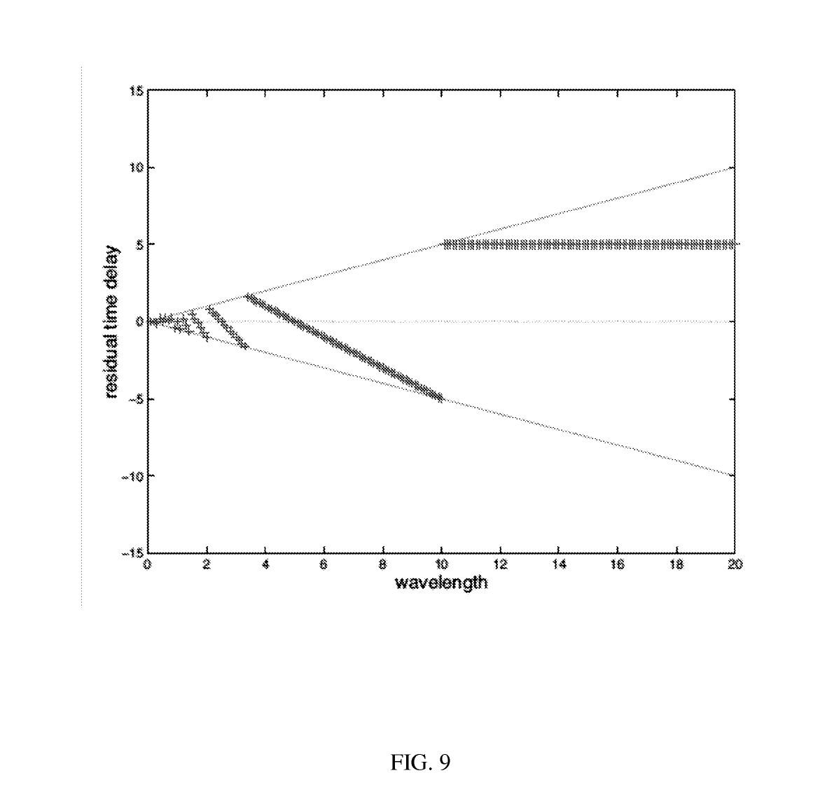

In one aspect, a Plot of Δti versus λi, may be used, and look for horizontal linear behavior (FIG. 9).

### ii) Peak-to-Nadir Time Delay

In one aspect, while phase difference is computed peak-to-peak, it is conceivable that a wave's peak would naturally be caused by the nadir of another wave; a drop in blood pressure causing an increase in EMG in the legs, for example.

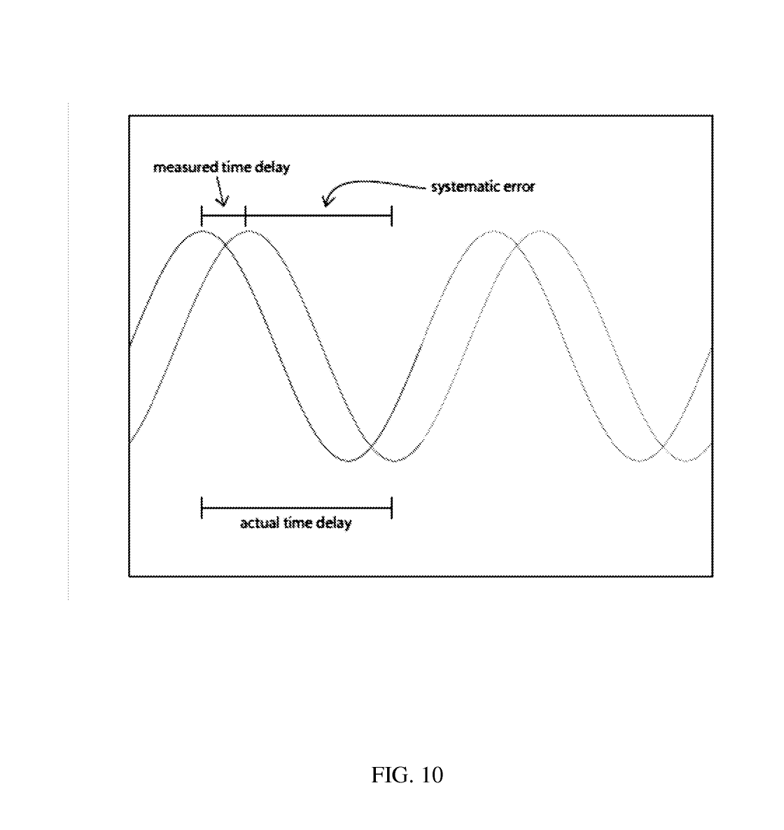

In one such embodiment, this issue may be easily dealt with by noticing that the systematic error (shown in FIG. 10) is exactly λ/2. The procedure is then similar to that of the first section using:

$$\frac{\cdot Δ {φ}_{i} \cdot {λ}_{i}}{2 π} - \frac{{λ}_{i}}{2} = Δ {t}_{i} .$$

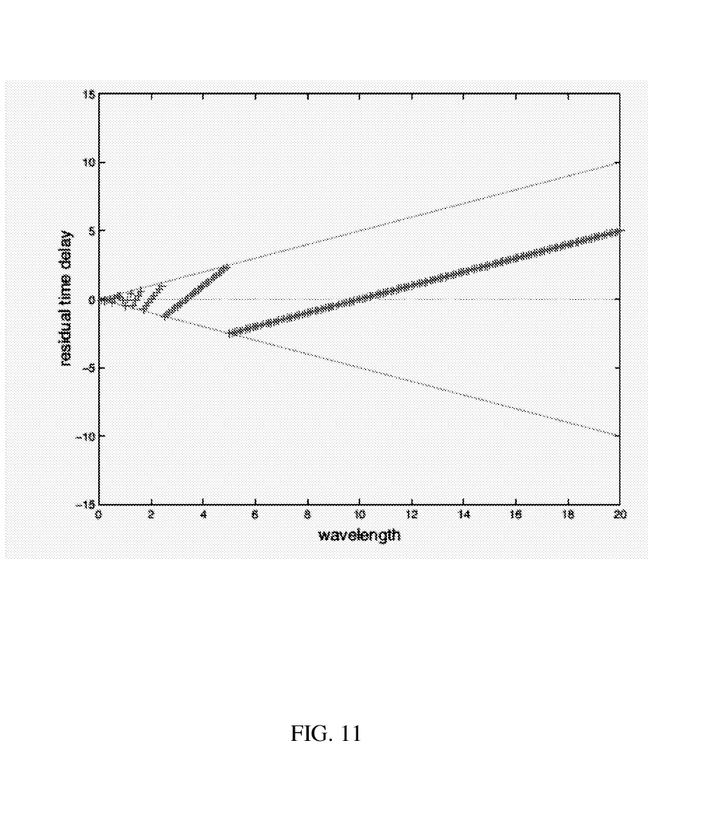

In one such case, for >Δt, Δtᵢ=(λᵢ/2)−Δt, which results in a line parallel to the range of the definition, and whose y-intercept is Δt, such as shown in FIG. 11.

8. Obtaining Overall Time Delay(s) from the Funnel Graph.

In one aspect, a funnel graph is searched through for consecutive sequences of more than 4 points {xi, yi} whose mean absolute time difference from its mean time was less than 5% of its mean time were identified as statistically relevant lines, i.e. consecutive sequences {Δtᵢ} such that:

$$\frac{{\sum }_{i}^{N > 3} \vert Δ {t}_{i} - \overline{Δ {t}_{i}} \vert}{N} < 0.05 \cdot \overline{Δ {t}_{i}} .$$

In one such embodiment, the graph can be divided up into multiple regions depending on the time delays expected. This procedure can then be conducted independently in the specified regions. Lines with the maximum number of points are kept and the line with the highest mean gain is selected.

### 9. Determination of Causality

In one embodiment, based on the natural interaction of the signals the position of the line on the funnel graph represents the direction, or causality, of the signal. A positive value indicates A→B whereas a negative value represents B→A; where A was the original input value of the analysis. The mean value of the gain for the EVENT represented from the time delay line indicates the strength of the interaction at that time delay.

### 10. Example of Time-Delay for Baroreflex

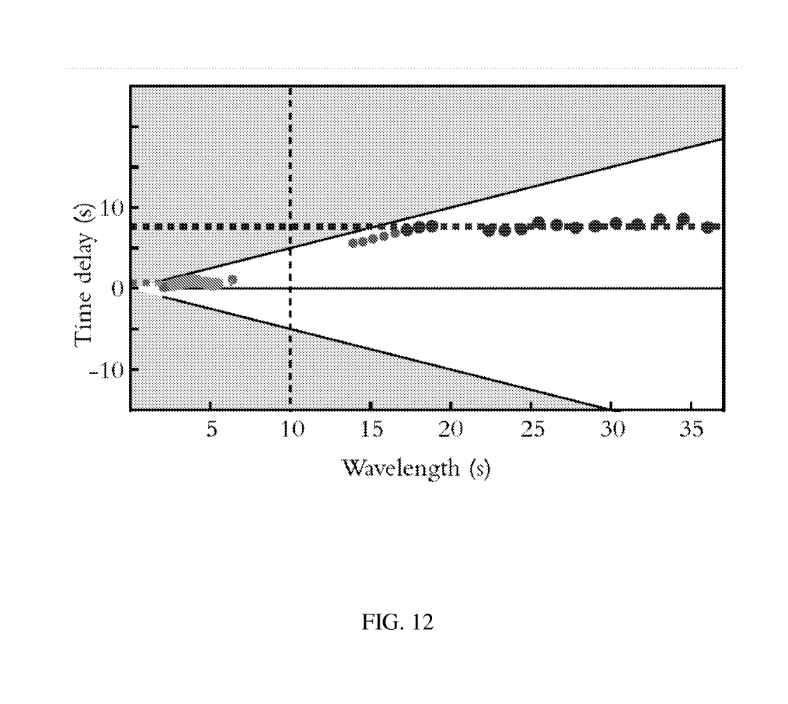

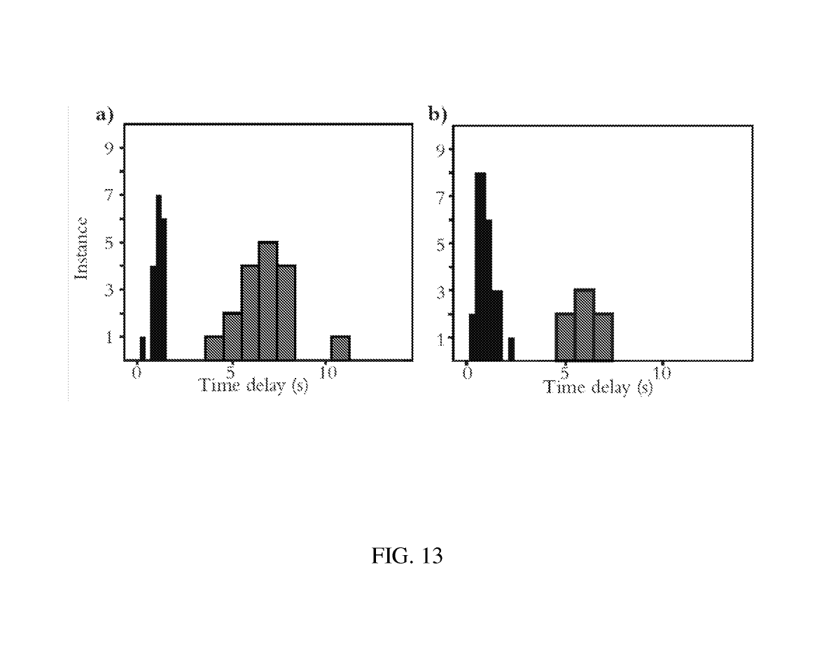

In one embodiment of the present invention, the processing technique techniques developed and described herein can be used in any paired system with multiple control delays (not only for the cardio-postural model). In a particular embodiment, an example of its application to a system with two delays is shown in FIGS. 12 and 13. In one such embodiment, the baroreflex is known to be coordinated by both components of the autonomic nervous system, parasympathetic (PNS) and sympathetic (SNS). The response times (time delay) for these have been measured invasively in animals and in humans and is on the order of 0.8 s (PNS) and 5-10 s (SNS). This technique can be used if one already has a residual time delay versus wavelength and is interested in testing the hypothesis that a time delay causal relationship exists between the two phenomena involved.

The exemplary embodiments herein described are not intended to be exhaustive or to limit the scope of the invention to the precise forms disclosed. They are chosen and described to explain the principles of the invention and its application and practical use to allow others skilled in the art to comprehend its teachings.

As will be apparent to those skilled in the art in light of the foregoing disclosure, many alterations and modifications are possible in the practice of this invention without departing from the scope thereof.

Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment. Further, the described features, structures, or characteristics of the present disclosure may be combined in any suitable manner in one or more embodiments. In this Detailed Description of the Invention, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure.

The scope of the present disclosure fully encompasses other embodiments and is to be limited, accordingly, by nothing other than the appended claims, wherein any reference to an element being made in the singular is intended to mean “one or more”, and is not intended to mean “one and only one” unless explicitly so stated. All structural and functional equivalents to the elements of the above-described preferred embodiment and additional embodiments are hereby expressly incorporated by reference and are intended to be encompassed by the present claims Moreover, no requirement exists for an apparatus or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, are also encompassed by the present disclosure.

1. A cardio-postural assessment system comprising:
a non-invasive force platform comprising at least one force sensor;
at least one pair of electromyography sensors;
at least one pair of electrocardiography sensors;
at least one ballistocardiography sensor;
an electronic processor connected to said at least one force sensor, said electromyography sensors, said electrocardiography sensors, and said ballistocardiography sensor and adapted to receive signals therefrom, to calculate a center of pressure from signals received from said force sensor, an electromyogram from signals received from said electrocardiography sensors, an electrocardiogram from signals received from said electrocardiography sensors, and a ballistocardiogram from signals received from said ballistocardiography sensor, and further to analyze at least a plurality of said center of pressure, electromyogram, electrocardiogram and ballistocardiogram to generate at least one output signal corresponding to a cardio-postural physiological model.

2. The cardio-postural assessment system according to claim 1, wherein said ballistocardiography sensor comprises at least one of a force sensor and accelerometer attached to said force platform and oriented in a vertical direction with respect to said force platform.

3. The cardio-postural assessment system according to claim 1, wherein said force platform additionally comprises first and second force sensors oriented to provide perpendicular horizontal directions with respect to said force platform.

4. The cardio-postural assessment system according to claim 1, wherein said electromyography sensors comprise a plurality of electromyographic electrodes situated on a surface of said force platform and adapted to interface with a foot of a human patient.

5. The cardio-postural assessment system according to claim 1, wherein said at least one pair of electrocardiography sensors comprise at least one of hand, foot, leg and torso electrocardiography electrodes.

6. The cardio-postural assessment system according to claim 1, wherein said electronic processor additionally comprises an electromyography circuit adapted to receive signals from said electromyographic sensors and to calculate an electromyogram.

7. The cardio-postural assessment system according to claim 1, wherein said electronic processor additionally comprises an electrocardiography circuit adapted to receive signals from said electrocardiographic sensors and to calculate an electrocardiogram.

8. The cardio-postural assessment system according to claim 1, wherein said electronic processor additionally comprises a ballistocardiography circuit adapted to receive signals from said ballistocardiographic sensors and to calculate a ballistocardiogram.

9. The cardio-postural assessment system according to claim 1, wherein said electronic processor is additionally adapted to calculate a coherence wavelet analysis of at least a plurality of said center of pressure, electromyogram, electrocardiogram and ballistocardiogram, and to output at least one parameter corresponding to a cardio-postural physiological model.

10. The cardio-postural assessment system according to claim 1, wherein said system comprises a single diagnostic unit.

11. The cardio-postural assessment system according to claim 1, wherein said system comprises a portable diagnostic system adapted for carrying by an operator.

12. The cardio-postural assessment system according to claim 1, additionally comprising at least one display, wherein said display is adapted to show said at least one output signal.

13. The cardio-postural assessment system according to claim 1, additionally comprising a network interface adapted to connect to at least one computer network and to transmit said at least one output signal to said at least one computer network.

14. The cardio-postural assessment system according to claim 1, wherein said electronic processor comprises at least one of: analog and digital circuits and combinations thereof.

15. The cardio-postural assessment system according to claim 1, wherein said electronic processor is additionally adapted to record said signals from at least one of said force, electromyography, electrocardiography and ballistocardiography sensors.

16. A method for non-invasive cardio-postural assessment, comprising:
receiving signals from force, electromyography, electrocardiography and ballistocardiography sensors in communication with a subject under assessment;
calculating a center of pressure from signals received from said force sensor, an electromyogram from signals received from said electrocardiography sensors, an electrocardiogram from signals received from said electrocardiography sensors, and a ballistocardiogram from signals received from said ballistocardiography sensor;
analyzing at least a plurality of said center of pressure, electromyogram, electrocardiogram and ballistocardiogram to define discrete interaction events between signals and interaction strength of said interaction events;
analyzing said interaction events to determine time overlapping pairs of interaction events;
analyzing a plurality of said time overlapping pairs of interaction events to determine a degree of phase lock correlation between said pairs of interaction events within at least one frequency band;
determining a residual time delay from a phase difference of said pairs of interaction events for a plurality of single wavelengths;
determining an overall time delay for said pairs of interaction events from a consecutive sequence of said residual time delays over a plurality of said wavelengths;
determining a causality between said interaction events for each said pair of interaction events from said overall time delay for said pair; and
determining a strength of interaction between each said pair of interaction events from a maximum mean gain of said time delay for said pair of interaction events.

17. The method for non-invasive cardio-postural assessment according to claim 16, additionally comprising:
outputting at least one of said time delay, causality and strength of interaction between at least one pair of cardio-postural parameters of a cardio-postural model to an operator.

18. The method for non-invasive cardio-postural assessment according to claim 16, wherein said analyzing at least a plurality of said center of pressure, electromyogram, electrocardiogram and ballistocardiogram to define discrete interaction events between signals comprises discrete time, phase and gain analysis of said signals using at least one time-frequency analysis to define discrete interaction events.

19. The method for non-invasive cardio-postural assessment according to claim 18, wherein said at least one time-frequency analysis comprises at least one of a wavelet coherence analysis and a Hilbert transform analysis to determine phase lock regions comprising said discrete interaction events.

20. The method for non-invasive cardio-postural assessment according to claim 16, wherein at least one of said analyzing a plurality of said time overlapping pairs of interaction events to determine a degree of phase lock correlation between said pairs of interaction events within at least one frequency band, said determining a residual time delay from a phase difference of said pairs of interaction events for a plurality of single wavelengths, and said determining an overall time delay for said pairs of interaction events from a consecutive sequence of said residual time delays over a plurality of said wavelengths, comprises a graphical analysis method.
